(19) United States
(12) Patent Application Publication
(10) Pub. No.: US 2014/0187972 A1
(43) Pub. Date: Jul. 3, 2014

(54) Guidewire Devices and Methods

(71) Applicant: Volcano Corporation, San Diego, CA (US)

(72) Inventor: David H. Burkett, Temecula, CA (US)

(73) Assignee: Volcano Corporation, San Diego, CA (US)

(21) Appl. No.: 14/139,484

(22) Filed: Dec. 23, 2013

Related U.S. Application Data
(60) Provisional application No. 61/747,758, filed on Dec. 31, 2012.

(51) Int. Cl. A61B 5/00; A61B 5/02

(52) U.S. Cl. CPC A61B 5/6851; A61B 5/02
USPC 600/481; 29/825

(57) ABSTRACT

Guidewire devices and methods are disclosed. In a preferred embodiment, a guidewire including a flexible element having a first center point, a stiffening core wire comprising at least three wires collectively having a second center point and extending from a proximal section to a distal section within the flexible element, wherein the flexible element has an outer diameter of about 0.035 inches or less, and wherein the first and second center points are within about 0.001 inches of each other, is included. Methods of making and/or assembling such guidewire devices are also provided.

### CROSS-REFERENCE TO RELATED APPLICATIONS

The present application claims priority to and the benefit of U.S. Provisional Patent Application No. 61/747,758, filed Dec. 31, 2012, which is hereby incorporated by reference in its entirety.

### TECHNICAL FIELD

The present disclosure relates to guidewire devices and methods. In particular, it relates to a guidewire device including a stiffening core of at least three wires wherein the device has an outer diameter of about 0.035 inches or less, and methods for making and assembling such devices.

### BACKGROUND

Often, guidewire devices are used to measure pressure within a patient's vessel, visualize the inner lumen of the vessel, and/or otherwise obtain data related to the vessel, e.g., a blood vessel. It may be desired to include pressure sensors, imaging elements, and/or other electronic, optical, or electro-optical components in a guidewire, however, assembly of such guidewires containing electronic components is complex due the space needed for the conductors or communication lines of the electronic component(s), the stiffness of the rigid housing containing the electronic component(s), and/or other limitations associated with providing the functionality of the electronic components in the limited space available within a guidewire. Further, due to its small diameter, in many instances the proximal connector portion of the guidewire (i.e., the connector(s) that facilitate communication between the electronic component(s) of the guidewire and an associated controller or processor) is fragile and prone to kinking, which can destroy the functionality of the guidewire. Guidewires tend to include a relatively stiff core wire extending substantially the length of the device that forms the backbone of the guidewire. The core wire has sufficient column stiffness to transfer compressive pushing force applied to the proximal end to the distal end to facilitate advancement of the guidewire within a vessel. The guidewire core also has a diameter that tends to limit the internal space for additional components.

Accordingly, there remains a need for improved guidewire devices and methods, particularly those adapted to include one or more electronic, optical, or electro-optical components.

### SUMMARY

Embodiments of the present disclosure are directed to guidewire devices and methods.

In a first aspect, the disclosure encompasses a guidewire that includes a flexible element having a first center point, a stiffening core wire including at least three wires collectively having a second center point and extending from a proximal section to a distal section within the flexible element, wherein the flexible element has an outer diameter of about 0.035 inches or less, and wherein the first and second center points are within about 0.001 inches of each other. In a preferred embodiment, the first and second center points are co-axial within the flexible element.

In a preferred embodiment, each of the at least three wires each has a center point that forms a vertex of a first triangle about the second center point. In another preferred embodiment, each wire has an insulating coating disposed thereover sufficient to electrically isolate each from the other. In another preferred embodiment, at least one, preferably two, and more preferably all three of the wires are conductors. In another preferred embodiment, each of the at least three wires includes a stainless steel core having a conductive coating disposed thereon, and wherein the at least three wires are joined to each other.

In another preferred embodiment, a gap in the insulating coating on each of the at least three wires is positioned at a different point in the proximal direction to facilitate electrical connection to a connector. In a more preferred embodiment, the guidewire further includes a soldered component disposed between each gap and an opposing portion of the flexible element. In another embodiment, a gap in the insulating coating on each of the at least three wires exists adjacent the flexible element at the same co-axial radius to facilitate electrical connection to a connector. In this embodiment, each gap is preferably separated from the other two by an insulating spacer.

In another embodiment, the guidewire further includes at least three non-conductive core guides each having a core center point that forms a vertex of a first triangle about the third center point, wherein the at least three wires are disposed about the at least three non-conductive core guides and each of the at least three wires has a center point that forms a vertex of a second triangle about the second center point, and the second and third center points are within about 0.001 inches of each other. In a preferred embodiment, the first, second, and third center points are co-axial within the flexible element.

In another embodiment, each of the at least three wires includes a conductive core and an insulating coating disposed thereon, and the at least three non-conductive core guides are joined to each other. In a preferred embodiment, a gap in the insulating coating on each of the at least three wires is positioned at a different point in the proximal direction to facilitate electrical connection to a connector. In another preferred embodiment, a gap in the insulating coating on each of the at least three wires exists adjacent the flexible element at the same co-axial radius to facilitate electrical connection to a connector.

In another embodiment, the guidewire further includes a non-conductive core guide that has at least three recessed portions oriented longitudinally along a length thereof and that is at least substantially co-axial within the flexible element, wherein the at least three wires are disposed at least partially within the at least three recessed portions. In a preferred embodiment, the three recessed portions are arranged as far apart around the non-conductive core guide as possible. In another preferred embodiment, the at least three wires are each electrically connected to a conductive lead in a proximal section via an arc-shaped conductive connector disposed therebetween. In a more preferred embodiment, the guidewire further includes an insulator disposed between each arc-shaped conductive connector so that the insulators and conductive connectors encircle the stiffening core wire.

In a second aspect, the disclosure encompasses a guidewire, including a flexible element, a stiffening core wire including a first wire, and second and third wires that are at least substantially the same in radius with both being smaller than the first wire, and extending from a proximal section to a distal section within the flexible element, wherein the flexible element has an outer diameter of about 0.035 inches or less, and wherein each of the first, second, and third wires includes an insulating coating disposed thereon sufficient to prevent electrical interconnection therebetween.

In a third aspect, the disclosure encompasses a guidewire, including a first flexible element, a second flexible element coupled to the first flexible element in a position proximal to the first flexible element, a third flexible element coupled to the second flexible element in a position proximal to the second flexible element, a distal core extending within the first flexible element, a mounting structure positioned within the second flexible element and fixedly secured to the distal core, the mounting structure configured to have at least one component selected from the group of components consisting of an electronic component, an optical component, an electro-optical component mounted thereto, at least one electronic, optical, or electro-optical component mounted to the mounting structure, a stiffening core wire fixedly attached to the mounting structure and extending proximally from the mounting structure through the second and third flexible elements, and at least three wires having a proximal and a distal section, therein the distal section of at least one of the at least three wires is coupled to the at least one electronic component and the proximal section of the at least three wires is coupled to at least one connector, wherein each of the three wires has a center point that forms a vertex of a triangle having a first center point, wherein the first, second, and third flexible elements have an outer diameter of no more than about 0.035 inches or less and each having a second center point; and

wherein the first and second center points are within about 0.001 inches of each other.

In one embodiment, the second flexible element includes a ribbon coil. In another embodiment, each of the at least three wires has a thickness of no more than about 0.0022 inches. In yet a further embodiment, the at least three wires are configured so that only a single wire extends distally from the second flexible element into the first flexible element.

In a fourth aspect, the disclosure encompasses a method of assembling a guidewire, including providing a polymer tubing having a first center point, providing at least three wires having a proximal portion and a distal portion, wherein a distal portion of at least one of the wires is coupled to at least one component selected from the group of components consisting of an electronic component, an optical component, and an electro-optical component, electrically coupling a proximal portion of the at least three wires to conductive bands adjacent a proximal portion of the polymer tubing, wherein the at least three wires are positioned about a second center point, and wherein the first and second center points are within about 0.001 inches of each other.

In a fifth aspect, the disclosure encompasses a guidewire adapted to be inserted into a tubular structure of a patient, which guidewire includes a core wire extending from a proximal portion of the guidewire to a distal portion of the guidewire, and a tubular flexible member surrounding at least a portion of the core wire, wherein at least a portion of the core wire is formed by at least three wires extending along a longitudinal axis of the core wire.

Additional aspects, features, and advantages of the present disclosure will become apparent from the following detailed description.

### BRIEF DESCRIPTION OF THE DRAWINGS

The present disclosure is best understood from the following detailed description when read with the embodiments, or examples, illustrated in the accompanying figures. It is emphasized that various features are not necessarily drawn to scale. It will nevertheless be understood that no limitation of the scope of the invention is thereby intended. Any alterations and further modifications in the described embodiments, and any further applications of the principles of the invention as described herein are contemplated as would normally occur to one of ordinary skill in the art to which the invention relates.

Illustrative embodiments of the present disclosure, which form part of the present specification, will be described with reference to the accompanying drawings, of which:

FIG. 1 is a diagrammatic side view of a guidewire system according to an exemplary embodiment of the present disclosure.

FIG. 2 is a diagrammatic perspective view of a guidewire according to an exemplary embodiment of the present disclosure.

FIG. 3 illustrates a stylized version of a guidewire incorporating a composite core wire construct according to one aspect of the present disclosure.

FIG. 4 is a diagrammatic cross-sectional longitudinal view taken along line 3-3 of a proximal portion of the device shown in FIG. 3, according to an embodiment of the present disclosure.

FIG. 5 is a diagrammatic cross-sectional longitudinal view taken along the same cross-section as line 3-3 in FIG. 3 in an alternative embodiment of the present disclosure.

FIG. 6 is a diagrammatic cross-sectional longitudinal view taken along the same cross-section as line 3-3 in FIG. 3 in an alternative embodiment of the present disclosure.

FIG. 7 is a diagrammatic cross-sectional side view of a guidewire device according to another aspect of the present disclosure.

FIG. 8 is a diagrammatic cross-sectional longitudinal view taken along the same cross-section as line 7-7 in FIG. 7, but illustrates a different core embodiment of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one of ordinary skill in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

The present disclosure relates to a guidewire device having a split-conductor arrangement, preferably also including a split-core arrangement. The split-core arrangement, e.g., using three or more stiffening core wires in various embodiments, can permit a larger amount of stiffening core cross-section to be used, an increased conductive cross-section, or both. The split-core wire disclosed herein has sufficient column stiffness to transfer compressive pushing force applied to the proximal end to the distal end to facilitate advancement of the guidewire within a vessel. Without being bound by theory, it is believed that the split-core wire advantageously provides sufficient column stiffness in a longitudinal direction as a completely circular unitary, core member, preferably while also increasing the cross-sectional area available for the conductors. Each of the embodiments disclosed herein can greatly increase the handling performance characteristics of the guidewire device of the present disclosure. Moreover, a split-core in some preferred embodiments tends to reduce or avoid prolapse of the guidewire during use.

As used herein, “flexible elongate member” or “elongate flexible member” includes any thin, long, flexible structure that can be inserted into a vessel of a patient, such as the vasculature. While the illustrated embodiments of the “flexible elongate members” of the present disclosure have a cylindrical profile with a circular cross-sectional profile that defines an outer diameter of the flexible elongate member, in other instances all or a portion of the flexible elongate members may have other geometric cross-sectional profiles (e.g., oval, rectangular, square, elliptical, etc.) or non-geometric cross-sectional profiles. Flexible elongate members include, for example, guidewires and catheters. In that regard, catheters may or may not include a lumen extending along its length for receiving and/or guiding other instruments. If the catheter includes a lumen, the lumen may be centered or offset with respect to the cross-sectional profile of the device. In one embodiment, a combination product including a split-core guidewire and a catheter containing at least a partial lumen adapted to receive a distal portion of the guidewire are provided.

In some embodiments, the flexible elongate member of the present disclosure includes one or more electronic, optical, or electro-optical components. For example, without limitation, a flexible elongate member may include one or more of the following types of components: a pressure sensor, a temperature sensor, an imaging element, an optical fiber, an ultrasound transducer, a reflector, a minor, a prism, an ablation element, an RF electrode, a conductor, and/or combinations thereof. Generally, these components are configured to obtain data related to a vessel or other portion of the anatomy in which the flexible elongate member is disposed. Often the components are also configured to communicate the data to an external device for processing and/or display. In some aspects, embodiments of the present disclosure include imaging devices for imaging within the lumen of a vessel, including both medical and non-medical applications. However, some embodiments of the present disclosure are particularly suited for use in the context of human vasculature. Imaging of an inner vessel surface, particularly the interior walls of human vasculature can be accomplished by a number of different techniques, including ultrasound (often referred to as intravascular ultrasound (“IVUS”) when used in connection with vascular imaging, as well as intracardiac echocardiography (“ICE”)) and optical coherence tomography (“OCT”). In other instances, infrared, thermal, or other imaging modalities are utilized.

The electronic, optical, and/or electro-optical components of the present disclosure are often disposed within a distal portion of the flexible elongate member. As used herein, “distal portion” of the flexible elongate member includes any portion of the flexible elongate member from the mid-point to the distal tip. As a guidewire might not provide sufficient room along much of its length for such electronic components, the flexible elongate members may include a housing portion at the distal portion adapted to receive one or more electronic components. Such housing portions can be tubular structures attached to the distal portion of the elongate member. Some flexible elongate members are tubular and have one or more lumens in which the electronic components can be positioned within the distal portion.

The electronic, optical, and/or electro-optical components and the associated communication lines are sized and shaped to allow for the diameter of the flexible elongate member to be very small. For example, the outside diameter of the flexible elongate member, such as the guidewire, containing one or more electronic, optical, and/or electro-optical components as described herein are from about 0.0007″ (0.0178 mm) to about 0.118″ (3.0 mm), with some particular embodiments having outer diameters of approximately 0.014″ (0.3556 mm), approximately 0.018″ (0.4572 mm), and approximately 0.035″ (0.889 mm). As such, the flexible elongate members incorporating the electronic, optical, and/or electro-optical component(s) of the present application are suitable for use in a wide variety of lumens within a human patient besides those that are part or immediately surround the heart, including veins and arteries of the extremities, renal arteries, blood vessels in and around the brain, and other lumens.

The terms “connected,” “secured,” and variations thereof, as used herein, includes direct connections, such as being welded (e.g., stitch welded), glued, melt bonded, soldered, coated, or otherwise fastened directly to, on, within, etc. another element, as well as indirect connections where one or more elements are disposed between the connected elements.

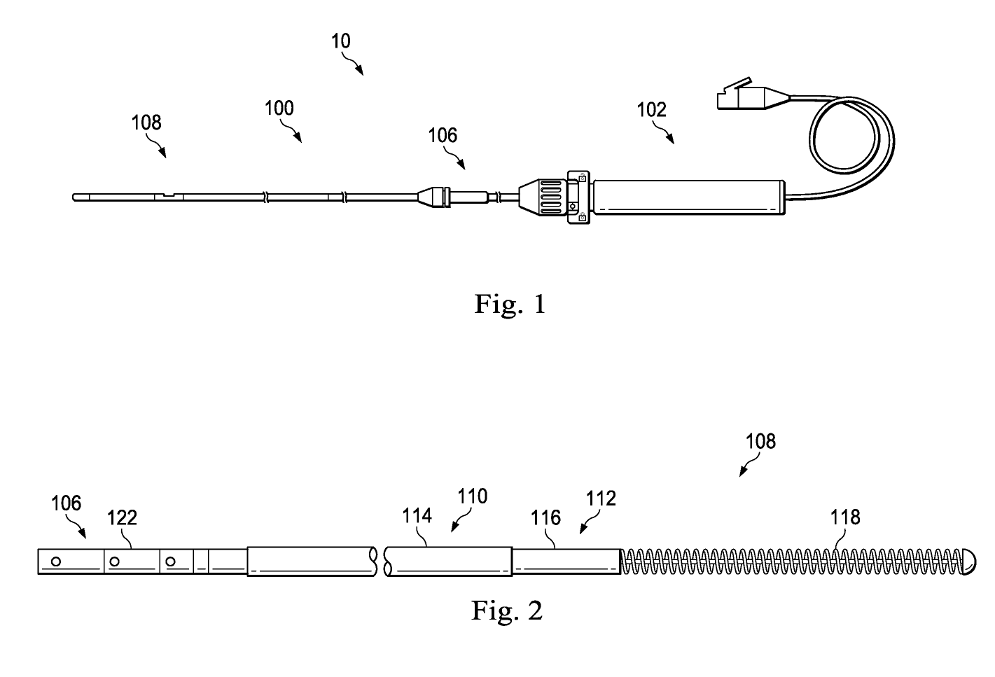

FIG. 1 shows an exemplary guidewire system 10 consistent with the principles disclosed herein. The guidewire system 10 in this embodiment is configured to sense or detect a physiological characteristic of one or more conditions of a patient. For example, it may detect or sense a characteristic of the vessel through which it has been introduced. In one embodiment, the guidewire system 10 has pressure sensing capabilities. The guidewire system 10 includes a guidewire 100 and a connector 102 disposed at the end of the guidewire 100. The connector 102 in this example in FIG. 1 is configured to communicate with the guidewire 100, serve as a grippable handle to enable a surgeon or other medical professional to easily manipulate the proximal end of the guidewire 100, and connect to a console or further system (not shown) with a modular plug. Accordingly, since the guidewire 100 is configured to detect physiological environmental characteristics, such as pressure in an artery for example, data or signals representing the detected characteristics may be communicated from the guidewire 100, through the connector 102, to a console or other system for processing. In this embodiment, the connector 102 is configured to selectively connect to and disconnect from the guidewire 100. In some embodiments, the guidewire system 10 is a single-use device The guidewire 100, in the embodiment shown, is selectively attachable to the connector 102 and includes a proximal portion 106 connectable to the connector 102 and a distal portion 108 configured to be introduced to a patient during a surgical procedure.

The guidewire 100 is shown in greater detail in FIG. 2, which shows the entire guidewire 100. The guidewire includes a hypotube 110, a sensor housing 112, a proximal polymer sleeve 114, a sensor assembly 116, a distal tip 118, and a proximal electrical interface 122.

The proximal electrical interface 122 in FIG. 2 is configured to electrically connect the sensor assembly 116 and the connector 102 to order to ultimately communicate signals to the processing system. In accordance with this, the electrical interface 122 is in electrical communication with the sensor assembly 116 and in this embodiment is configured to be received within the connector 102. The electrical interface may include a series of conductive contacts on its outer surface that engage and communicate with corresponding contacts on the connector 102.

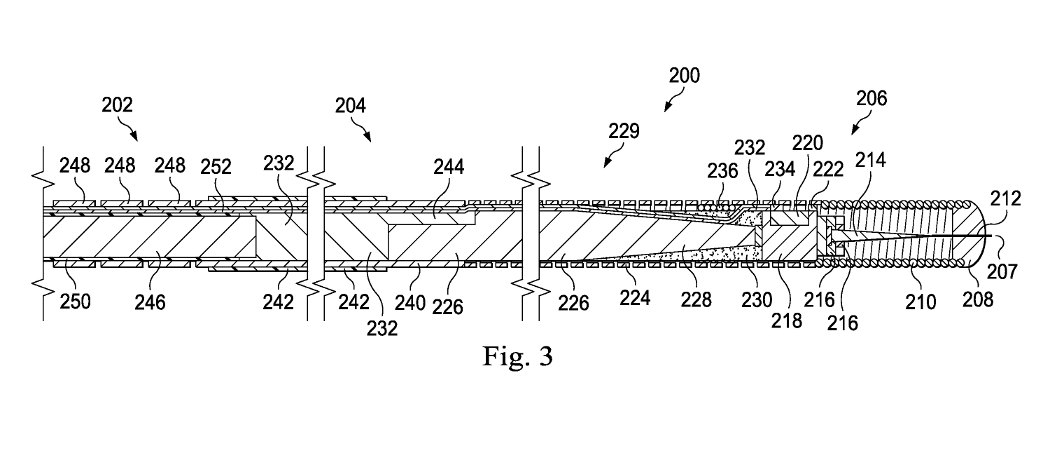

FIG. 3 illustrates a stylized version of a guidewire cross-section incorporating a composite core wire construct according to one aspect of the present disclosure. The guidewire device 200 is provided as an exemplary embodiment of the type of device into which the mounting structures, including the associated structural components and methods, described below with respect to FIGS. 3-8 can be implemented. It is understood, however, that no limitation is intended thereby and that the concepts of the present disclosure are applicable to a wide variety of guidewire devices, including those described in U.S. Pat. No. 7,967,762 and U.S. Patent Application Publication No. 2009/0088650, each of which is hereby incorporated by reference in its entirety.

As shown in FIG. 3, the device 200 includes a proximal portion 202, a middle portion 204, and a distal portion 206. Generally, the proximal portion 202 is configured to be positioned outside of a patient, while the distal portion 206 and a majority of the middle portion 204 are configured to be inserted into the patient, including within a human vessel such as the vasculature. In that regard, the middle portion 204 and/or distal portion 206 have an outer diameter from about 0.0007″ (0.0178 mm) to about 0.118″ (3.0 mm) in some embodiments, with some particular embodiments having an outer diameter of approximately 0.014″ (0.3556 mm), approximately 0.018″ (0.4572 mm), or approximately 0.035″ (0.889 mm). In the illustrated embodiment of FIG. 3, the middle and distal portions 204, 206 of the device 200 each have an outer diameter of 0.014″ (0.3556 mm).

As shown, the distal portion 206 of the device 200 has a distal tip 207 defined by an element 208. Features 207 and 208 and associated structures discussed below are optional and not required in most embodiments. In the illustrated embodiment, the distal tip 207 has a rounded profile. In some instances, the element 208 is radiopaque such that the distal tip 207 is identifiable under x-ray, fluoroscopy, and/or other imaging modalities when positioned within a patient. In some particular instances, the element 208 may be solder secured to a flexible element 210 and/or a flattened tip core 212. In that regard, in some instances the flexible element 210 is a coil spring. The flattened tip core 212 extends distally from a distal portion of a core 214. As shown, the distal core 214 tapers to a narrow profile as it extends distally towards the distal tip 207. In some instances, the distal core 214 is formed of, e.g., a stainless steel or Nitinol, which may be formed with a desired tapered profile or may be ground down to have a desired tapered profile. In some particular instances, the distal core 214 is formed of high tensile strength 304V stainless steel. In an alternative embodiment, the distal core 214 is formed by wrapping a stainless steel shaping ribbon around a Nitinol core.

In some embodiments, the distal core 214 is secured to a mounting structure 218 by mechanical interface, solder, adhesive, combinations thereof, and/or other suitable techniques as indicted by reference numerals 216. The mounting structure 218 is configured to receive and securely hold a component 220. In that regard, the component 220 is one or more of an electronic component, an optical component, and/or electro-optical component. For example, without limitation, the component 220 may be one or more of the following types of components: a pressure sensor, a temperature sensor, a flow sensor, an imaging element, an optical fiber, an ultrasound transducer, a reflector, a mirror, a prism, an ablation element, an RF electrode, a conductor, and/or combinations thereof.

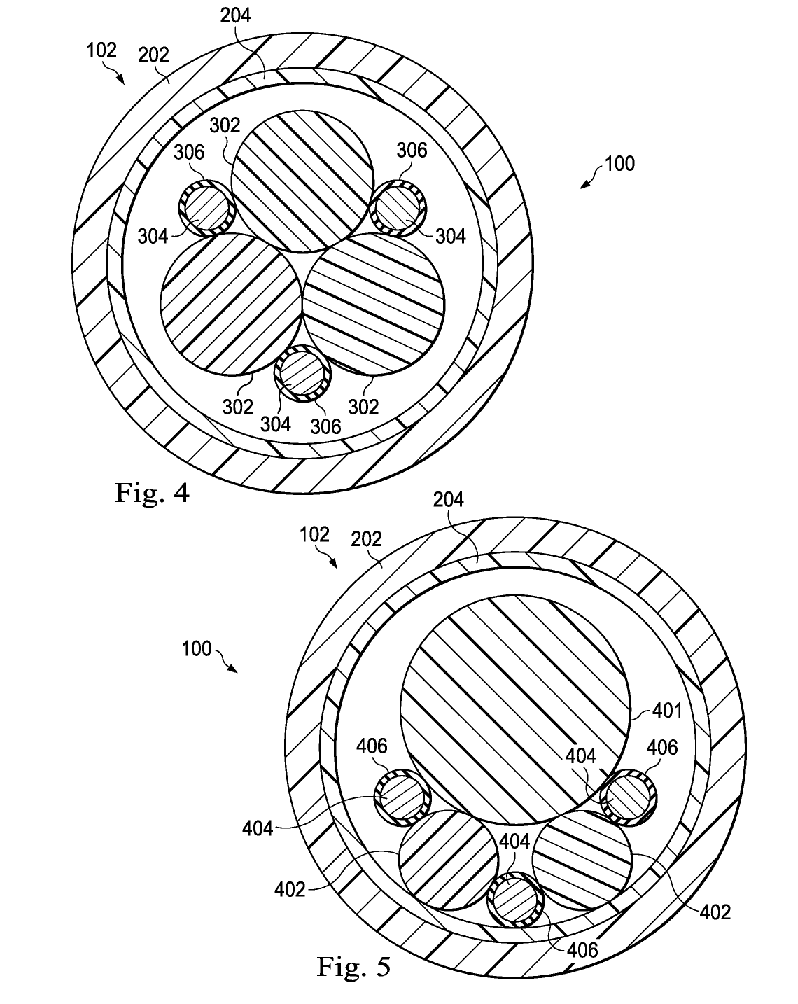

The mounting structure 218 shown in FIG. 3 is fixedly secured within the distal portion 206 of the device 200. As will be discussed below in the context of the exemplary embodiments of FIGS. 4-8, an electronic component 220 may be directly, or through a mounting structure 218 indirectly, fixedly secured to a core wire (i.e., a core running along the length of the mounting structure formed of a split core wires, split conductor, or both), flexible elements or other components surrounding at least a portion of the mounting structure (e.g., coils, polymer tubing, etc.), and/or other structure(s) of the device 200 positioned adjacent to the mounting structure 218. In the illustrated embodiment, the mounting structure is disposed at least partially within flexible element 210 and/or a flexible element 224 and secured in place by an adhesive or solder 222. In some embodiments, the mounting structure 218 is disposed entirely within flexible element 210 and/or flexible element 224. In some instances, the flexible elements 210 and 224 are flexible coils. In one particular embodiment, the flexible element 224 is ribbon coil covered with a polymer coating. For example, in one embodiment the flexible element 224 is a stainless steel ribbon wire coil coated with polyethylene terephthalate (PET). In another embodiment, the flexible element is a polyimide tubing that has a ribbon wire coil embedded therein. An adhesive is used to secure the mounting structure 218 to the flexible element 210 and/or the flexible element 224 in some implementations. Accordingly, in some instances the adhesive is urethane acrylate, cyanoacrylate, silicone, epoxy, and/or combinations thereof.

The mounting structure 218 may also be secured to a multi-part core 226 that extends proximally from the mounting structure towards the middle portion 204 of the device 200. In that regard, core 226 and optional distal core 214 are integrally formed in some embodiments such that at least one portion of a continuous core passes through the mounting structure. In the illustrated embodiment, a portion of the core 226 tapers as it extends distally towards mounting structure 218. However, in other embodiments the core 226 has a substantially constant profile along its length until one or more of the core wires terminates at a distal location that is proximal to the mounting structure 218, and typically between the intermediate zone 204 and the distal position where the tapering begins. In some instances, the stiffening core is ground down to have the desired tapered profile. Either one wire may be ground beyond the distal point where the other conductive wires terminate, or two or more wires (e.g., three wires) may collectively be ground down at the distal end. In other instances, all but one of the wires in core 226 terminate at a distal position and the remaining core wire is tapered to a narrower diameter at a more distal position adjacent or beyond the mounting structure 218. By tapering or reducing the number of core wires, the flexibility of the stiffening core can be increased to permit easier advancement of the guidewire in vivo.

In some implementations, the diameter or outer profile (for non-circular cross-sectional profiles) of core 226 and core 214 are the same. Like distal core 214, the core 226 is fixedly secured to the mounting structure 218. In some instances, solder and/or adhesive is used to secure the core 226 to the mounting structure 218. In the illustrated embodiment, solder/adhesive 230 surrounds at least a part of the portion 228 of the core 226. In some instances, the solder/adhesive 230 is the solder/adhesive 222 used to secure the mounting structure 218 to the flexible element 210 and/or flexible element 224, or to the tapered core 226 itself. In other instances, solder/adhesive 230 is a different type of solder or adhesive than solder/adhesive 222. In one particular embodiment, adhesive or solder 222 is particularly suited to secure the mounting structure 218 to flexible element 210, while solder/adhesive 230 is particularly suited to secure the mounting structure to flexible element 224.

A communication cable 232 extends along the length of the device 200 from the proximal portion 202 to the distal portion 206. In that regard, the distal end of the communication cable 232 is coupled to the component 220 at junction 234. The type of communication cable used is dependent on the type of electronic, optical, and/or electro-optical components that make up the component 220. In that regard, the communication cable 232 may include one or more of an electrical conductor, an optical fiber, and/or combinations thereof. Appropriate connections are utilized at the junction 234 based on the type of communication lines included within communication cable 232. For example, electrical connections are soldered in some instances, while optical connections pass through an optical connector in some instances. In some embodiments, the communication cable 232 is a trifilar structure, a bifilar structure, a single conductor (which may be a conductive core or a conductor separate from the core). Further, it is understood that all and/or portions of each of the proximal, middle, and/or distal portions 202, 204, 206 of the device 200 may have cross-sectional profiles as shown in FIGS. 2-5 of U.S. Provisional Patent Application No. 61/665,697 filed on Jun. 28, 2012, which is hereby incorporated by reference in its entirety.

Further, in some embodiments, the proximal portion 202 and/or the distal portion 206 incorporate spiral ribbon tubing as disclosed in U.S. Provisional Patent Application No. 61/665,697 filed on Jun. 28, 2012. In some instances, the use of such spiral ribbon tubing allows a further increase in the available lumen space within the device. For example, in some instances use of a spiral ribbon tubing having a wall thickness between about 0.001″ and about 0.002″ facilitates the use of a core wire having an outer diameter of at least 0.0095″ within a 0.014″ outer diameter guide-wire using a trifilar with circular cross-sectional conductor profiles. The size of the core wire can be further increased to at least 0.010″ by using a trifilar with conductors spaced out circumferentially around the core wire and extending longitudinally along the length of the core wire. In one embodiment, the use of a plurality of core wires having an increased diameter allows the use of materials having a lower modulus of elasticity than a standard stainless steel core wire (e.g., superelastic materials such as Nitinol or NiTiCo are utilized in some instances) without adversely affecting the handling performance or structural integrity of the guidewire and, in many instances, provides improvement to the handling performance of the guidewire, especially when a superelastic material with an increased core diameter (e.g., a core diameter of 0.0075″ or greater) is utilized within the distal portion 206.

The distal portion 206 of the device 200 also optionally includes at least one imaging marker 236. In that regard, the imaging marker 236 is configured to be identifiable using an external imaging modality, such as x-ray, fluoroscopy, angiograph, CT scan, MRI, or otherwise, when the distal portion 206 of the device 200 is positioned within a patient. In the illustrated embodiment, the imaging marker 236 is a radiopaque coil positioned around the tapered distal portion 228 of the core 226. Visualization of the imaging marker 236 during a procedure can give the medical personnel an indication of the size of a lesion or region of interest within the patient. To that end, the imaging marker 236 can have a known length (e.g., 0.5 cm or 1.0 cm) and/or be spaced from the element 218 by a known distance (e.g., 3.0 cm) such that visualization of the imaging marker 236 and/or the element 218 along with the anatomical structure allows a user to estimate the size or length of a region of interest of the anatomical structure. It is understood that a plurality of imaging markers 236 are utilized in some instances. In that regard, in some instances the imaging markers 236 are spaced a known distance from one another to further facilitate measuring the size or length of the region of interest.

As shown in FIG. 3, there is a transition from the main body in the middle portion 204 of the composite core wire 226 formed by the three individual stiffening core wire members to the tapering transition zone 229. In the illustrated embodiment, the transition is accomplished by the termination of individual stiffening core wire members prior to the termination of the transition zone 229. More specifically, as shown in FIG. 3, the lower core wire is narrowed as it extends distally within the transition region and then terminates before the end of the transition zone 229 while the upper core wire continues through the transition zone 229 substantially or completely unchanged in diameter. In this manner of stopping one or more the individual core wires making up the composite core 226, the desired transition in flexibility can be achieved.

The transition between the core 226 and the narrower diameter portion permits the communication cable 232 to separate into three or more conductors that extend proximally towards the proximal portion 202 through the channels, recessions, or other gap that exists between pairs of core wires 226 at a longitudinal position where they have not yet been tapered. For example, in the illustrated embodiment the transition adjacent core 226, the tapering begins at a proximal location within the flexible element 224, and in another at a proximal location within the flexible element 240, or in between the two. The flexible element 240 in the illustrated embodiment is a hypotube. In some particular instances, the flexible element is a stainless steel hypotube. Further, in the illustrated embodiment a portion of the flexible element 240 is covered with a coating 242. In that regard, the coating 242 is a hydrophobic coating in some instances. In some embodiments, the coating 242 is a polytetrafluoroethylene (PTFE) coating.

The proximal portion of core 226 is fixedly secured to the device, preferably in a series of connections along an inner wall of the device as it extends to the distal portion 206. In that regard, any suitable technique for securing the core wires 226 to one another may be used as disclosed herein, as well as to secure the core wires 226 to the device itself at least at one end, preferably both ends, and more preferably along its length between the proximal and distal positions. In some instances, the core wires 226 are soldered together. In some instances, an adhesive is used to secure the core wires 226 together. As shown in FIG. 3, a series of connections may be used to join adjacent stiffening wires to form a composite core wire. In some embodiments, combinations of structural interfaces, soldering, and/or adhesives are utilized to secure the core wires 226 together. In other instances, the core wires 226 are not fixedly secured to the distal tip 207, if present. For example, in some instances, the core wires 226 are fixedly secured to the hypotube 240, which helps to maintain the position of the core wires 226 particularly in relation to the flexible elongate body of the device 100 and the conductors that extend along the length of the guidewire.

In some instances, a proximal portion of the core 226 extends through at least a portion of the proximal portion 202 of the device 200. In one feature, the composite core wire, or a portion thereof, extends uninterrupted from the proximal end to the distal portion adjacent the sensor. In an alternative feature, the composite core wire may terminate proximally to the distal sensor region and a different distal core wire component may be coupled to the composite core wire. For example, in the illustrated embodiment, a plurality of conducting bands 248 are disposed concentrically around the core wires 246. In some instances, the conductive bands 248 are portions of a hypotube, while in others they have the same circumference. Proximal portions of the conductive communication cable 232 are separately coupled to the conductive bands 248. In that regard, in some instances each of the conductive bands 248 is preferably associated with a corresponding communication line of the communication cable 232. For example, in embodiments where the communication cable 232 consists of a trifilar, each of the three conductive bands 248 are connected to one of the conductors of the trifilar, for example by soldering each of the conductive bands to the respective conductor or by adhering each conductive band 248 to the respective conductor of the communication cable 232 with solder or another conductive connector. Where the communication cable 232 includes optical communication line(s), the proximal portion 202 of the device 200 includes an optical connector in addition to or instead of one or more of the conductive bands 248. An insulating layer or sleeve 250 preferably separates the conductive bands 248 from the core wires 246 and any separate split communication cable 232. In some instances, the insulating layer 250 is formed of polyimide. The insulating layer 250 may be ablated or otherwise disrupted (including at time of manufacture or assembly) to permit electrical contact, such as using a conductive adhesive like solder, between the wires of the communication cable 232 when needed.

As noted above, the proximal portion of core wires 226 are fixedly secured to the proximal portion of core wires 246. In that regard, any suitable technique for securing the cores 226, 246 to one another lengthwise may be used if they are separate stiffening wires. Preferably, however, continuous stiffening wires 246, 226 are used and extend from a proximal position 202 to a distal position 206 as shown. In some embodiments, at least one of the core wires 246 includes a structural feature that is utilized to couple the core wires together. In the illustrated embodiment, the core wires 246 include an extension 252 that extends around a distal portion of the core wires 246. Extension 252 may be an insulator to inhibit or prevent arcing of electricity between the core wires 246 and communication cable conductors 232, or between the core wires 246 or the communication cable conductors 232 and an inner wall or other surrounding structure of the device. In some instances, the core wires 246 are soldered together. In some instances, an adhesive is utilized to secure the core wires 246 together. In some embodiments, combinations of structural interfaces, soldering, and/or adhesives are utilized to secure the cores 246 together. In some embodiments, the core 246 is formed of a different material than the core 226. For example, in some instances the core 246 is formed of Nitinol and/or NiTiCo (nickel-titanium-cobalt alloy) and the core 226 is formed of stainless steel. In that regard, by utilizing a Nitinol core within the conductive bands 248 instead of a stainless steel, the likelihood of prolapse (or kinking) is greatly reduced because of the increased flexibility of the Nitinol core compared to a stainless steel core. In other instances, the core wires 226 and the core wires 246 are formed of the same material. In some instances the core wires 226 have a different profile than the core wires 246, such as a larger or smaller diameter and/or a non-circular cross-sectional profile. In other instances, core wires 226 and core wires 246 are made of the same material and/or have the same structure profiles with the same diameter.

Referring now to FIGS. 4-6, shown therein are various cross-sectional profiles of guidewire devices of the present disclosure that illustrate different stiffening core wire configurations shown at cross-section 3-3. In some embodiments, these include split-core wire techniques for extending communication pathways (e.g., electrical conductors and/or optical fibers) along the length of the device. In that regard, one of the major issues associated with existing functional guidewires is poor mechanical performance as compared to frontline guidewires. This performance loss is due in a large part to the typical design of the guidewires that severely limits the space available for the core or core wire due to the need to run the communication lines along the length of the device. As noted herein, for the sake of clarity and simplicity, the embodiments of FIGS. 4-6 include three core wires in various configurations. More specifically, in some embodiments of FIGS. 4-6 as further discussed below, these include three electrical conductors arranged as a trifilar. Existing trifilars are typically formed by three individual copper wires each wrapped with a color coded insulation material.

FIG. 4 illustrates a cross-sectional longitudinal view of a guidewire device according to an embodiment of the present disclosure. As noted, some features in cross-sectional FIGS. 4-6 and 8 are similar to those described above and may use the same reference numerals to refer to similar components; however, some of reference numerals may be the same as FIG. 3 but refer to different components than in FIG. 3 and this is true of all following discussion of the FIGS. below. The device 100 includes a flexible elongate member 102 having an outer wall 202 defining an outer boundary of the device 100 and an inner wall 204 defining a lumen for receiving additional components of the device 100 as discussed herein. It should be understood, however, that in various embodiments of the disclosure only a single wall is used to form the flexible elongate member 102 and that wall would thus account for both the inner and outer walls. In the illustrated embodiment, however, outer wall 202 has a circular cross-sectional profile. The stiffening core wires 302 are arranged in a closely packed configuration where their center points of their cores, or core center points, form the vertices of a triangle. In the depicted embodiment, the core wires 302 are non-conductive. The center point of the composite stiffening core is typically at least substantially co-axial with the center point of the entire flexible elongate member 102, and preferably these points are co-axial. In the illustrated cross-section, the outermost surfaces of the three individual stiffening wires 302 forming the composite core wire are shown spaced from the inner surface 204 for the purpose of illustration, however, it will be understood that the diameter of the composite core would likely be increased in practice (and in all embodiments disclosed herein) to more closely match the diameter of inner surface 204 to thereby increase the overall cross-section of the composite core wire and provide greater stiffness to the guidewire. The term “at least substantially,” referring to the center point overlap or displacement, it is meant they are preferably within about 0.001 inches, more preferably within about 0.0005 inches, of each other. Three conducting wires 304 are also included, and each of these has a single insulating coating 306 layer disposed thereover as depicted in this embodiment.

The three conducting wires in this embodiment are similar to a conventional trifilar arrangement, although they are preferably arranged so that the conducting wire center points form the vertices of a second triangle. The center point of the second triangle is preferably also at least substantially co-axial with the center point of the core wire triangle, the center point of the flexible elongate member 102, or both. Preferably, all three center points are at least substantially co-axial, or entirely co-axial.

For example, in this embodiment, the three conductive outer wires 304 are disposed within the lumen of the flexible elongate member 102 defined by the inner wall 204. Electrical connections or conductive wires of any embodiment of the present disclosure may be formed of any suitable conductive material including without limitation copper, copper alloy, silver, silver alloy, aluminum, gold, platinum/iridium alloy (e.g., 80/20), platinum-tungsten alloy, and/or any combinations thereof, or any of the foregoing may be plated over another less conductive material, such as stainless steel. Although different materials may be selected for each wire, preferably the same conductive materials are selected. An insulating layer 306 may be used and formed from any suitable insulating material, including without limitation polyimide, polyurethane, nylon, polyethylene, polypropylene, silicone rubber, fluoropolymers, and/or combinations thereof. Preferably, the coating 304 includes polyimide. Different insulating materials may be used on the different wires 304, but preferably the same materials are selected. In some embodiments, the insulating layers 306 are color coded or otherwise include markings or identifiers to facilitate identification of the corresponding conductor 304. An overcoat layer (not shown) may be formed over the three conductors 304 and insulating layers 306. For example, in some instances a portion of the space inside inner wall 204 is filled with an adhesive, such as one or more polymer components, epoxies, silicones, or combinations thereof. Suitable polymer components include without limitation those formed from one or more urethanes, cyanoacrylates, ethylenes (e.g., polyethylene terephthalate (PET)), acrylates, or any combinations thereof. A filler, or adhesive, is typically provided to secure components (e.g., conductive stiffening core wires, or conducting wires and non-conductive stiffening core wires) of the device 100 together, to minimize or prevent shifting of the components within the flexible elongate member 102, or both. The adhesive, when used, may extend along all or a portion of the length of the core wire 302, intermittently along the length as an insulator at certain joints and/or to help secure the conductive wires 304 to the stiffening core wires 302, etc. While the adhesive material is preferably any suitable thickness to fill any remaining space between the core wires and any separate conductors, in some embodiments the adhesive material may have a thickness from about 0.0001″ (0.0025 mm) to about 0.0005″ (0.0127 mm). It should be understood that all materials discussed herein, for example, the adhesive, the stiffening core wires, the insulating layer(s), the conductive wires, etc., are equally applicable for the same type of material regardless of which embodiment is being described herein.

Referring now to FIG. 5, shown therein is a cross-sectional longitudinal view of an device 100 according to another embodiment of the present disclosure. The device 100 includes a flexible elongate member 102 having an outer wall 202 defining an outer boundary of the device 100 and an inner wall 204 defining a lumen for receiving the split-conductor arrangement of the device 100 as discussed in greater detail herein. In the illustrated embodiment, the three stiffening core wires 401, 402 are sized differently. As depicted, core wire 401 is larger than the other two core wires 402, which may be useful in certain guidewire configurations. In this embodiment, a ground wire may be core wire 401 and other smaller conducting wires 402. The core wires 401, 402 and conducting wires 404, with insulating coating layers 406, still each have center points that form respective first and second triangles, however, these would be isosceles triangles rather than equilateral triangles as was possible when the core wires were sized the same. Thus, the center points of the triangles formed by the core wire center points and the conductive wire center points will be offset. Preferably, the size differential is selected so that the center points of the first and second triangles will be at least substantially co-axial. In addition, the use of differential diameters between the individual stiffening members 401, 402 forming the composite core wire more easily allows the distal portion of the guidewire to transition to alternative stiffnesses by the elimination of one or more of the individual stiffening core wire members. The change in stiffness by reducing the number of wires forming the composite core wire can allow the manufacture of the device while eliminating much of the grinding currently used to create the transition of the flexibility of the core wire as it extends toward the distal end. Alternatively or additionally, each wire forming the composite core wire can be selected with a desired stiffness to achieve the desired flexibility toward the distal end of the guidewire. In one exemplary embodiment (not shown), the larger core wire 401 and smaller core wires 402 are conductive and have an insulator disposed over the wires, such that conducting wires 404 with insulating layers 406 are not present.

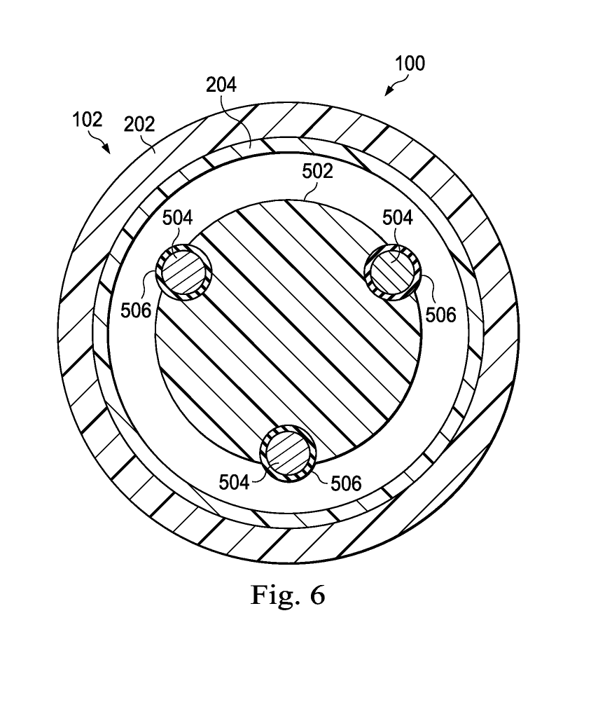

Referring now to FIG. 6, shown therein is a cross-sectional longitudinal view of a guidewire device 100 according to another embodiment of the present disclosure. The device 100 includes a flexible elongate member 102 having an outer wall 202 defining an outer boundary of the device 100 and an inner wall 204 defining a lumen for receiving additional components of the device 100 that are discussed in greater detail below. In the illustrated embodiment, the flexible elongate body 102 has a circular cross-sectional profile. As shown, the stiffening core 502 is a single structure having longitudinally extending recessions or grooves spaced about the circumference to receive three wires 504. In this embodiment, the stiffening core 502 is non-conductive and the three wires 504 are conductors over which an insulating layer 506 is disposed. The recessions may be of any shape or size sufficient to recess the spaced wires within a portion of the core 502 itself, and they typically run along substantially all, or all, the length of the core to the distal portion of the guidewire device 100 at least until the stiffening core 502 is tapered in one embodiment. For example, the recessions may be different shapes or the same, and may be circular (as shown), U- or V-shaped grooves or notches, square, etc. Typically, the recesses in the outer surface of the core 502 are sized and dimensioned to match the three wires 504 they will receive. The recesses are preferably sufficiently deep to recess at least a portion, preferably a predominant portion, of the wires 504. In one embodiment, the outermost radial point of the recessed wires 504 will be along the circumference of the core 502 (not shown).

Although not shown, it is possible for the central core 502 to be conductive, in which case an insulating layer is preferably disposed over the outer surface thereof. In that embodiment, the three spaced wires 504 may be non-conductive stiffening core members and need not have an insulating coating disposed thereover. While the recesses are evenly spaced around the circumference as shown based on the number of wires 504 included, it is possible for these to be unequally spaced. In such an embodiment, the center point of the triangle formed by the center of each of the three wires 504 is preferably at least substantially co-axial with the center point of the stiffening core 502. As depicted, however, the recesses and associated wires 504 are spaced at 120°.

The stiffening core is preferably made of stainless steel, and is as large as possible to fill the available space inside the inner wall of the flexible elongate member while still leaving space, in certain embodiments where the core (optionally including three core wires) is non-conductive, for three conductive wires. One preferred embodiment includes high tensile strength 304V stainless steel in the stiffening core, while another includes MP-35N stainless and another includes Nitinol, and yet a further includes NiTiCo (nickel-titanium-cobalt alloy). While nickel-titanium alloys are often overlooked because welding may be more difficult than with stainless steel materials, such alloys may be useful in certain stiffening core embodiments, such as shown in FIG. 6. When only a single core wire is included or where no welding is used to intermittently or continuously connect the core wires, the weldability of the core wire material is less relevant. Moreover, the core material may also be less relevant in embodiments where a Nitinol core is coated with a conducting layer or an insulating layer.

In some instances, the stiffening core is ground down to have the desired tapered profile. Either one wire may be ground beyond the distal point where the other conductive wires will terminate, or the three wires may collectively be ground down at the distal end.

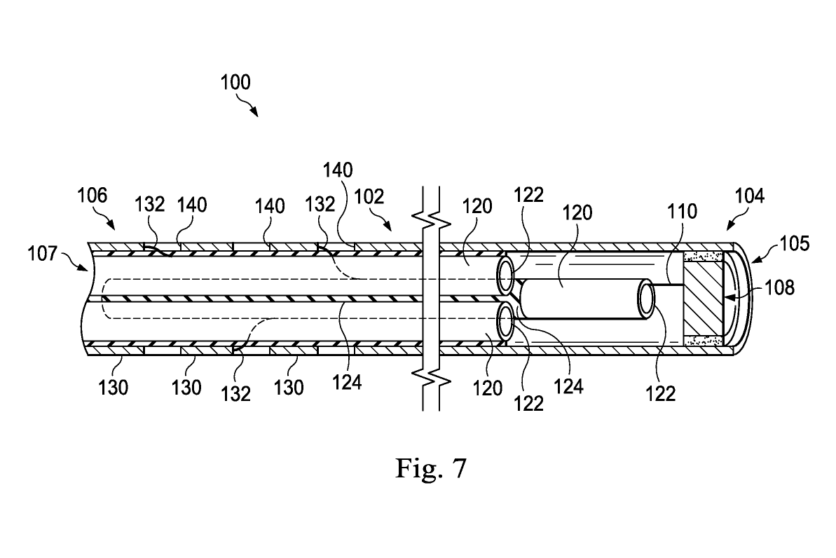

Referring now to FIG. 7, shown therein is a portion of an guidewire device 100 according to an embodiment of the present disclosure. In that regard, the guidewire device 100 includes a flexible elongate member 102 having a distal portion 104 adjacent a distal end 105 and a proximal portion 106 adjacent a proximal end 107. A component 108 may be positioned within the distal portion 104 of the flexible elongate member 102 proximal of the distal end 105. Generally, the component 108 is representative of one or more optional electronic, optical, or electro-optical components. In that regard, the component 108 may be a pressure sensor, a temperature sensor, a flow sensor, an imaging element, an optical fiber, an ultrasound transducer, a reflector, a mirror, a prism, an ablation element, an RF electrode, a conductor, and/or combinations thereof. The specific type of component or combination of components can be selected based on an intended use of the guidewire device. In some instances, the component 108 is positioned less than about 10 cm, preferably less than about 5, or less than about 3 cm from the distal end 105. In some instances, the component 108 may be positioned within a housing of the flexible elongate member 102 and secured thereto. In that regard, the housing may be a separate component secured to the flexible elongate member 102. In other instances, the housing may be integrally formed as a part of the flexible elongate member 102. A mounting structure may be used at the distal portion 104 in FIG. 7 to fixedly secure any electronic component 108 to the flexible elongate body 102. This mounting structure can include an adhesive or solder.

The guidewire 100 also includes a connector 110 or series of connectors extending from the proximal portion 106 of the device to the distal portion 104. The connector 110 is configured to facilitate communication of signals between any optional sensing component(s) 108 and the proximal end 107 of the guidewire device 100, such as to facilitate communication of data obtained by the sensing component(s) 108 to another device, such as a computing device or processor. Accordingly, in some embodiments the connector 110 is an electrical conductor. In such instances, the connector 110 includes a plurality of wires that extend along the length of the flexible elongate member 102 and are electrically coupled to the sensing component(s) 108. In some instances, the connector 110 is configured to provide a physical connection to another device, either directly or indirectly. In other instances, the connector 110 is configured to facilitate wireless communication between the guidewire device 100 and another device. Generally, any current or future developed wireless protocol(s) may be utilized. In yet other instances, the connector 110 facilitates both physical and wireless connection to another device. In one embodiment, at least one of the connectors 110 is an optical fiber extending from the distal portion to the proximal portion of the guidewire. It should be understood that the description and details relating to FIG. 7 may be equally applicable to certain aspects of FIGS. 1-6, as well.

In the depicted embodiment, the guidewire 100 includes a stiffening core wire that is split into three wires 120. These wires may be conductive or non-conductive. When they are non-conductive, the three wires provide a stiffening core wire to facilitate positioning of the guidewire in vivo in a patient when in use. In the illustrated embodiment, however, the three wires 120 are conductive and each is coated with an insulator 122. In another embodiment (not shown), one or more intermediate layers may be included on each of the three wires 120, such as where the wire is non-conductive. A preferred intermediate layer in this embodiment is a conductive coating, which is disposed between, e.g., a non-conductive wire and an insulator 122 deposited as a coating over a portion of the underlying conductive portion. The insulator 122 may be disposed only where the three wires 120 are closest to minimize electrical conduction between them, however, preferably the wires 120 are each covered by insulator 122 over at least about 65%, preferably about 75%, and more preferably about 90% of the circumference. In a more preferred embodiment, the insulators 122 completely surround each wire 120 as shown to minimize or prevent any electrical arcing.

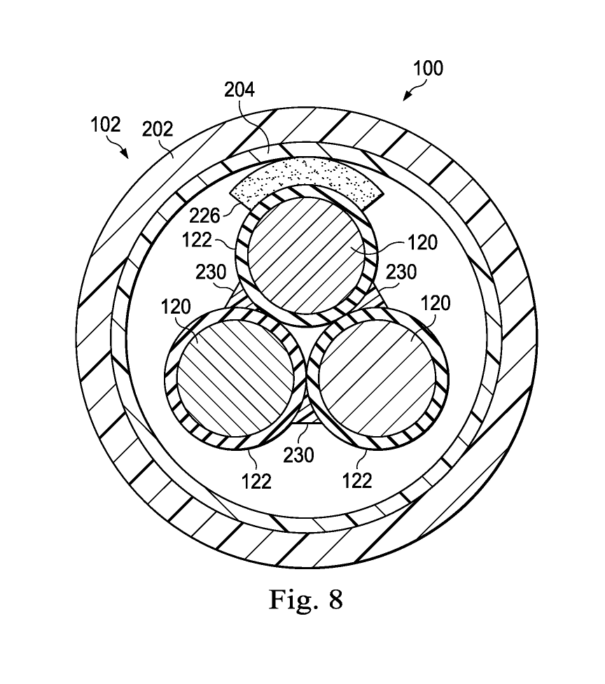

While in one embodiment the three core wires 120 are simply sandwiched into the available space inside the flexible elongate member 102 adjacent to each other and held in place by the flexible elongate member 102, or alternatively a filler wicked into any remaining space after assembly, the three wires 120 are preferably connected to each other as shown in FIG. 8. This connecting can be achieved by any suitable fastening device 124, including physical or chemical adherent(s), etc. Preferably, the three wires 120 are secured to each other as shown in FIG. 8 by a technique that will not disrupt the insulation surrounding the core wires 120. This assembly is preferably achieved before the three wires 120 are inserted inside the flexible elongate member 102. The fastening can occur at a series of discrete points along the length of the three wires 120, or along at least substantially all or all of the length of the three wires 120 where they are adjacent, as shown. It should be understood that when the wires are conductive, the connection occurs between the insulator 122 or other coating rather than the core wires.

When the stiffening core wires 120 are conductive, either in the core wires or a conductive intermediate layer 122, electrical connection in the proximal portion 106 is typically desired. As shown in FIG. 7, conductive bands 130 are alternated with insulating bands 140 around the stiffening core wires 120. Electrical connectors 132 may be positioned between the conductive bands 130 and the three wires 120, so as to electrically connect to the three wires 120 at different distal locations in the proximal portion 106. In this manner, each of the three wires 120 has a distinct electrical connector 132 to connect to its own conductive band 130 that electrically connects to an external device (not shown). In a preferred embodiment, as shown, solder or another electrically connective material can be disposed to facilitate such electrical connections and to minimize or avoid the risk of an electrical connector 132 breaking or coming loose from a conductive band 130 or a conductive core wire 120. In various embodiments, any insulator 122 may be ablated or otherwise formed so as to leave a gap sufficient to electrically connect the conducting wires of the core to each respective conductive band 130.

As shown, only one of the composite core wires 120 extends into the distal portion 104. In another embodiment (not shown), all three core wires 120 may extend into the distal portion with one, two, or three of them gradually tapering to a smaller diameter as they extend distally. The tapering of the core wire(s) 120 may occur adjacent to or in the distal portion 104, which permits the full diameter stiffening core wires 120 to more easily connect to the inside of the flexible elongate member 102 in the proximal portion 106. Generally, any number of electrical conductors, optical pathways, and/or combinations thereof can extend along the length of the flexible elongate member 102 between the connector 110 and the component 108. In some instances, between one and ten electrical conductors and/or optical pathways extend along the length of the flexible elongate member 102 between the connector 110 and the component 108. For the sake of clarity and simplicity, the embodiments of the present disclosure described below include three electrical conductors. However, it is understood that the total number of communication pathways and/or the number of electrical conductors and/or optical pathways is different in other embodiments and may include three, four, five, six, seven, eight, nine, ten, or even more conductor wires that extend from a proximal position to the connector 110, which itself can be formed of an equivalent (or different) number of conductor wires to connect to one or more electronic components 108. More specifically, the number of communication pathways and the number of electrical conductors and optical pathways extending along the length of the flexible elongate member 102 is determined by the desired functionality of the component 108 and the corresponding elements that define component 108 to provide such functionality.

Referring more specifically to FIG. 8, shown therein is a cross-sectional longitudinal view of a guidewire device according to an embodiment of the present disclosure. As noted, some features are similar to those described above and, therefore, the same reference numerals have been used to refer to similar components. The device 100 includes a flexible elongate member 102 having an outer wall 202 defining an outer boundary of the device 100 and an inner wall 204 defining a lumen for receiving additional components of the device 100 as discussed herein. In the illustrated embodiment, outer wall 202 has a circular cross-sectional profile. The outer wall 202 has a diameter of about 0.0007″ (0.0178 mm) to about 0.118″ (3.0 mm) in some embodiments, with some particular embodiments having an outer diameter of approximately 0.014″ (0.3556 mm), approximately 0.018″ (0.4572 mm), and approximately 0.035″ (0.889 mm). In some embodiments, the outer wall 202 has a constant profile along all or a majority of its length. In other embodiments (not depicted), there is only a single layer wall. In some embodiments, at the least the portions of the flexible, elongate member 102 that are intended to be disposed within the patient have a constant profile (or at least tapered/gradual transitions between portions with different outer profiles, preferably tapering to smaller diameter portions in a more distal direction along the device 100) to minimize or avoid potential injury to the patient while moving the device 100 through the patient. Further, it is recognized that the composition of the outer wall 202 may change along the length of the device in some instances.

As shown in FIG. 8, this embodiment depicts the stiffening core wire including at least three wires 120 that are conductive and coated in an insulator 122. As discussed herein, it should be understood that insulator 122 may not be included and the three wires 120 may simply provide a stiffening core to the guidewire device 100, or that one or more intermediate layers may be included between the three wire cores 120 and insulator 122, even though these are not depicted. The stiffening core wires 120 may be inert, and the intermediate layer on each may be a conductive material including without limitation those conductive materials described herein. An intermediate conductive layer (not shown) may be included by plating, such as electroplating, the core wires, and then the insulator 122 may be disposed thereon. Moreover, an adhesive 226, which can be conductive or non-conductive depending on the configuration of the stiffening core wire, may be included between the inner wall 204 and the outermost portion of each of the three wires 120. This adhesive 226 can be solder to facilitate or maintain electrical connections, or as shown a non-conductive adhesive can be provided into any open space after the stiffening core wire is assembled and inserted. The adhesive is preferably disposed between each of the three wires 120 and the inner wall 204, although this is only shown with respect to a single wire 120. A connection 230 is also shown between the three wires 120 in FIG. 8. Depicted are connections 230 positioned between two wires 120 and extending between pairs of wires, although a connection could be disposed internally between all three wires 120 in an additional or alternative embodiment (not shown).

One exemplary embodiment includes a stainless steel core of 304V stainless in three stiffening core wires coated with silver, which then has a polyimide coating disposed thereon. In this embodiment, such as shown in FIG. 8, no additional set of conducting wires is required.

Methods of forming the devices herein are also included as described herein and as would be readily understood by those of ordinary skill in the art based on the devices and systems, and related guidance, provided herein.

The term “about,” as used herein, should generally be understood to refer to both numbers in a range of numerals. Moreover, all numerical ranges herein should be understood to include each whole integer within the range.

Persons of ordinary skill in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A guidewire, comprising:
a flexible element having a first center point;
a stiffening core wire comprising at least three wires collectively having a second center point and extending from a proximal section to a distal section within the flexible element;
wherein the flexible element has an outer diameter of about 0.035 inches or less; and
wherein the first and second center points are within about 0.001 inches of each other.

2. The guidewire of claim 1, wherein the first and second center points are co-axial within the flexible element.

3. The guidewire of claim 1, wherein each of the at least three wires has a center point that forms a vertex of a first triangle about the second center point, and each has an insulating coating disposed thereover sufficient to electrically isolate each from the other.

4. The guidewire of claim 3, wherein at least one of the at least three wires is conductive.

5. The guidewire of claim 3, wherein each of the at least three wires comprises a stainless steel core having a conductive coating disposed thereon, and wherein the at least three wires are joined to each other.

6. The guidewire of claim 3, wherein a gap in the insulating coating on each of the at least three wires is positioned at a different point in the proximal direction to facilitate electrical connection to a connector.

7. The guidewire of claim 6, further comprising a soldered component disposed between each gap and an opposing portion of the flexible element.

8. The guidewire of claim 1, which further comprises at least three non-conductive core guides each having a core center point that forms a vertex of a first triangle about the third center point, wherein the at least three wires are disposed about the at least three non-conductive core guides and each of the at least three wires has a center point that forms a vertex of a second triangle about the second center point, and the second and third center points are within about 0.001 inches of each other.

9. The guidewire of claim 8, wherein the first, second, and third center points are co-axial within the flexible element.

10. The guidewire of claim 8, wherein each of the at least three wires comprises a conductive core and an insulating coating disposed thereon, and the at least three non-conductive core guides are joined to each other.

11. The guidewire of claim 10, wherein a gap in the insulating coating on each of the at least three wires is positioned at a different point in the proximal direction to facilitate electrical connection to a connector.

12. The guidewire of claim 10, wherein a gap in the insulating coating on each of the at least three wires exists adjacent the flexible element at the same co-axial radius to facilitate electrical connection to a connector.

13. The guidewire of claim 1, which further comprises a non-conductive core guide that has at least three recessed portions oriented longitudinally along a length thereof and that is at least substantially co-axial within the flexible element, wherein the at least three wires are disposed at least partially within the at least three recessed portions.

14. The guidewire of claim 13, wherein the three recessed portions are arranged as far apart around the non-conductive core guide as possible.

15. The guidewire of claim 13, wherein the at least three wires are each electrically connected to a conductive lead in a proximal section via an arc-shaped conductive connector disposed therebetween.

16. The guidewire of claim 15, which further comprises an insulator disposed between each arc-shaped conductive connector so that the insulators and conductive connectors encircle the stiffening core wire.

17. The guidewire of claim 1, wherein the at least three wires include a first wire, and second and third wires that are at least substantially the same in radius with both being smaller than the first wire.

18. The guidewire of claim 17, wherein each of the first, second, and third wires comprises an insulating coating disposed thereon sufficient to prevent electrical interconnection therebetween.

19. A guidewire, comprising:
a first flexible element;
a second flexible element coupled to the first flexible element in a position proximal to the first flexible element;
a third flexible element coupled to the second flexible element in a position proximal to the second flexible element;
a distal core extending within the first flexible element;
a mounting structure positioned within the second flexible element and fixedly secured to the distal core, the mounting structure configured to have at least one component selected from the group of components consisting of an electronic component, an optical component, and an electro-optical component mounted thereto;
at least one electronic, optical, or electro-optical component mounted to the mounting structure; a stiffening core wire fixedly attached to the mounting structure and extending proximally from the mounting structure through the second and third flexible elements;
at least three wires having a proximal and a distal section, therein the distal section of at least one of the at least three wires is coupled to the at least one electronic component and the proximal section of the at least three wires is coupled to at least one connector, wherein each of the three wires has a center point that forms a vertex of a triangle having a first center point;
wherein the first, second, and third flexible elements have an outer diameter of no more than about 0.035 inches or less and each having a second center point; and
wherein the first and second center points are within about 0.001 inches of each other.

20. A method of assembling a guidewire, comprising:
providing a polymer tubing having a first center point;
providing at least three wires having a proximal portion and a distal portion, wherein a distal portion of at least one of the wires is coupled to at least one component selected from the group of components consisting of an electronic component, an optical component, and an electro-optical component; and
electrically coupling a proximal portion of the at least three wires to conductive bands adjacent a proximal portion of the polymer tubing,
wherein the at least three wires are positioned about a second center point; and
wherein the first and second center points are within about 0.001 inches of each other.
